(19) United States
(12) Patent Application Publication
(10) Pub. No.: US 2025/0339733 A1
(43) Pub. Date: Nov. 6, 2025

(54) INFORMATION PROCESSING APPARATUS, METHOD, AND SYSTEM

(71) Applicant: CaTe Inc., Tokyo (JP)

(72) Inventor: Kazuhiro TERASHIMA, Tokyo (JP)

(73) Assignee: CaTe Inc., Tokyo (JP)

(21) Appl. No.: 19/269,234

(22) Filed: Jul. 15, 2025

Related U.S. Application Data
(63) Continuation of application No. PCT/JP2024/000118, filed on Jan. 9, 2024.

(30) Foreign Application Priority Data
Feb. 7, 2023 (JP) JP2023-016495

(51) Int. Cl. A63B 24/00; G16H 20/30; G16H 50/30

(52) U.S. Cl. CPC A63B 24/0003; A63B 24/0075; G16H 20/30; G16H 50/30

(57) ABSTRACT

An information processing apparatus includes processing circuitry configured to: estimate a first exercise load amount when a first user is performing a target exercise event, based on sensing data relating to the first user; determine a first individual index of the first user of an exercise load amount for the target exercise event by performing a predetermined calculation on the first exercise load amount; and calculate a first parameter representing a characteristic of the first user relating to an exercise tolerance, based on the first individual index and a reference value of the exercise load amount for the target exercise event.

This application is based upon and claims the benefit of priority from the prior Japanese Patent Application No. 2023-016495, filed Feb. 7, 2023 and PCT Patent Application No. PCT/JP2024/000118, filed Jan. 9, 2024, the entire contents of which are incorporated herein by reference.

### FIELD

Embodiments described herein relate generally to an information processing apparatus, a method, and a system.

### BACKGROUND

Cardiac rehabilitation aims to help patients with heart disease regain their physical strength and self-confidence, return to comfortable family life and social life, and prevent recurrence of heart disease or re-hospitalization through a comprehensive activity program that includes exercise therapy. The focus of exercise therapy is aerobic exercise, such as walking, jogging, cycling, aerobics, etc. For safer and more effective aerobic exercise, it is preferable that patients exercise at an intensity near their own anaerobic threshold (AT).

The anaerobic threshold is an example of an evaluation index of exercise tolerance and corresponds to the point of change in cardiopulmonary status, i.e., the exercise intensity near the boundary between aerobic exercise and anaerobic exercise. The anaerobic threshold is generally determined by a CPX test (cardiopulmonary exercise test) in which exhaled gas is collected and analyzed while the test subject is subjected to a progressive exercise load. In the CPX test, the anaerobic threshold is determined based on the results measured by exhaled gas analysis (e.g., oxygen uptake, carbon dioxide emissions, tidal volume, respiration rate, minute volume, or a combination thereof). According to the CPX test, in addition to the anaerobic threshold, the peak oxygen uptake, which corresponds to the exercise intensity near the maximum exercise tolerance, can be determined.

Conventional system discloses determining whether or not a ventilatory threshold (VT) has been reached based on pulse information of a subject and adjusting the exercise load of an exercise providing device in accordance with the result of the determination.

In the technical idea described in the conventional system, in general, feedback control is performed such that the exercise load amount approaches the ventilatory threshold (VT). However, the load amount for the person who performs exercise depends not only on the type of exercise, but also on the body function and daily physical condition of the person. Therefore, even if such a technical idea is applied to exercise therapy, it is not possible to obtain information for determining how much exercise load amount is applied to the patient when the patient performs a designated exercise event, or what exercise event should be recommended to the patient in order to achieve the designated exercise load amount.

An object of the present disclosure is to provide information for planning or guiding exercise therapy, or a signal indicating a body function of a user.

### BRIEF DESCRIPTION OF THE DRAWINGS

FIG. 1 is a block diagram showing a configuration of an information processing system of the present embodiment.

FIG. 2 is a block diagram showing a configuration of a client apparatus of the present embodiment.

FIG. 3 is a block diagram showing a configuration of a server of the present embodiment.

FIG. 4 is a block diagram showing a configuration of a wearable device of the present embodiment.

FIG. 5 is an explanatory diagram of an aspect of the present embodiment.

FIG. 6 shows a data structure of an exercise event database of the present embodiment.

FIG. 7 shows a data structure of a user profile database of the present embodiment.

FIG. 8 shows a data structure of a parameter log database of the present embodiment.

FIG. 9 is a flowchart of exercise event recommendation processing of the present embodiment.

FIG. 10 shows a screen example displayed in the exercise event recommendation processing of the present embodiment.

FIG. 11 is a flowchart of parameter monitoring processing of the present embodiment.

FIG. 12 shows a data structure of a training data set that can be used in the present embodiment.

### DETAILED DESCRIPTION

In general, according to an embodiment, an information processing apparatus comprising processing circuitry configured to: estimate a first exercise load amount when a first user is performing a target exercise event, based on sensing data relating to the first user; determine a first individual index of the first user of an exercise load amount for the target exercise event by performing a predetermined calculation on the first exercise load amount; and calculate a first parameter representing a characteristic of the first user relating to an exercise tolerance, based on the first individual index and a reference value of the exercise load amount for the target exercise event.

Hereinafter, an embodiment of the present invention will be described based on the drawings. Note that, in the drawings illustrating the embodiment, the same constituent elements are denoted by the same reference numeral in principle, and repeated descriptions thereof will be omitted.

### (1) Configuration of Information Processing System

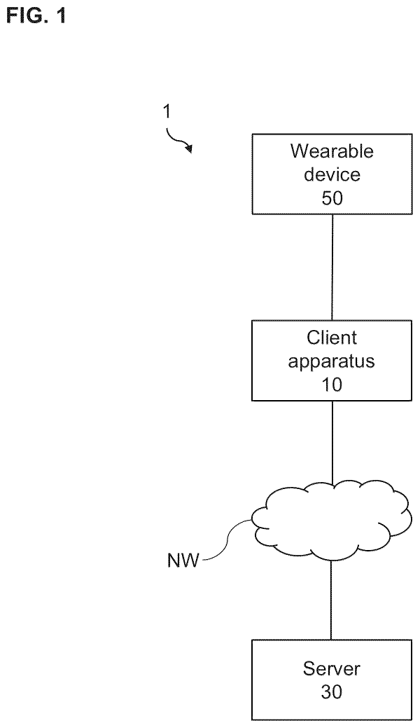

A configuration of an information processing system will be described. FIG. 1 is a block diagram showing a configuration of the information processing system of the present embodiment.

As shown in FIG. 1, an information processing system 1 includes a client apparatus 10, a server 30, and a wearable device 50.

Here, the number of client apparatuses 10 and wearable devices 50 varies depending on the number of users, for example. Accordingly, the number of each of the client apparatuses 10 and the wearable devices 50 may be two or more. Further, a terminal of a person who plans or guides exercise therapy can also be included in the information processing system 1. The person who plans or guides exercise therapy may include, for example, medical personnel (e.g., doctors, nurses, pharmacists, physical therapists, occupational therapists, clinical laboratory technicians), nutritionists, or trainers.

The client apparatus 10 and the server 30 are connected via a network (e.g., Internet or intranet) NW.

The client apparatus 10 and the wearable device 50 are connected through a wireless channel using, for example, the Bluetooth (registered trademark) technology.

The client apparatus 10 is an example of an information processing apparatus that transmits a request to the server 30. The client apparatus 10 is, for example, a smartphone, a tablet terminal, or a personal computer.

The server 30 is an example of an information processing apparatus that provides a response to the client apparatus 10 in response to a request transmitted from the client apparatus 10. The server 30 is, for example, a server computer.

The wearable device 50 is an example of an information processing device that can be worn on the user's body (e.g., arm).

### (1-1) Configuration of Client Apparatus

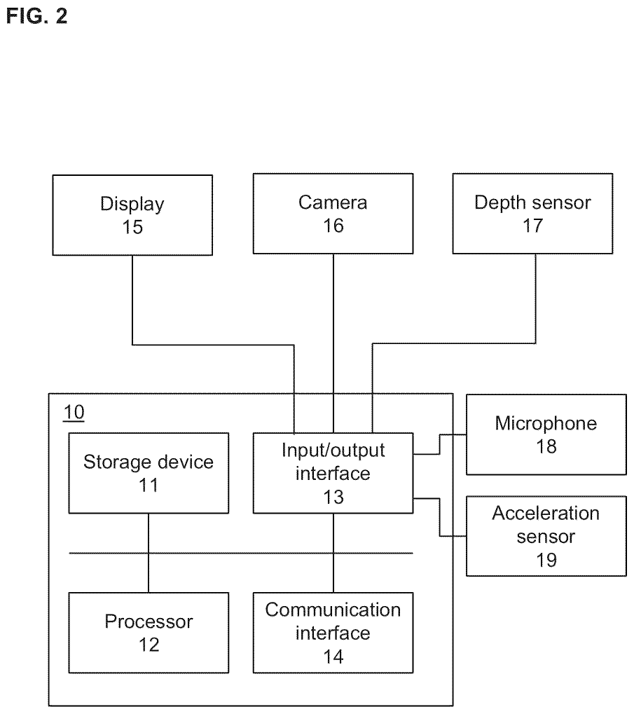

A configuration of the client apparatus will be described. FIG. 2 is a block diagram showing a configuration of the client apparatus of the present embodiment.

As shown in FIG. 2, the client apparatus 10 includes a storage device 11, a processor 12, an input/output interface 13, and a communication interface 14. The client apparatus 10 is connected to a display 15, a camera 16, a depth sensor 17, a microphone 18, and an acceleration sensor 19.

The storage device 11 is configured to store programs and data. The storage device 11 is, for example, a combination of a read only memory (ROM), a random access memory (RAM), and a storage (such as a flash memory or a hard disk).

The programs include, for example, the following programs.
Operating system (OS) program
Program for an application that executes information processing (e.g., a web browser, a therapeutic application, a rehabilitation application, or a fitness application)

Here, the target disease of the therapeutic application or the rehabilitation application is, for example, a disease, such as heart disease, lifestyle-related disease (hypertension, diabetes, dyslipidemia, hyperlipidemia), and obesity, for which exercise may contribute to the improvement of the symptoms.

The data includes, for example, the following data.
Database referred to in information processing
Data obtained by executing information processing (i.e., results of executing information processing)

The processor 12 is a computer that implements the functions of the client apparatus 10 by activating programs stored in the storage device 11. The processor 12 is, for example, at least one of the following.
Central processing unit (CPU)
Graphic processing unit (GPU)
Application specific integrated circuit (ASIC)
Field programmable gate array (FPGA)

The input/output interface 13 is configured to acquire information (such as a user instruction, an image, or a sound) from an input device connected to the client apparatus 10 and output information (such as an image or a command) to an output device connected to the client apparatus 10.

The input device is, for example, a camera 16, a depth sensor 17, a microphone 18, an acceleration sensor 19, a keyboard, a pointing device, a touch panel, a sensor, or a combination thereof.

The output device is, for example, a display 15, a speaker, or a combination thereof.

The communication interface 14 is configured to control communication between the client apparatus 10 and external apparatuses (such as another client apparatus 10, the server 30, and the wearable device 50).

Specifically, the communication interface 14 may include a module (e.g., a WiFi module, a mobile communication module, or a combination thereof) for communication with the server 30. The communication interface 14 may include a module (e.g., a Bluetooth module) for communication with the wearable device 50.

The display 15 is configured to display an image (still image or video). The display 15 is, for example, a liquid crystal display or an organic EL display.

The camera 16 is configured to pick up an image and generate an image signal.

The depth sensor 17 is, for example, a light detection and ranging (LIDAR). The depth sensor 17 is configured to measure a distance (depth) from the depth sensor 17 to a surrounding object (e.g., a user).

The microphone 18 is configured to receive a sound wave and generate a sound signal. The microphone 18 is preferably placed in the vicinity of the user's body (in particular, a respiratory organ), for example as an earphone microphone.

The acceleration sensor 19 is configured to detect acceleration.

### (1-2) Configuration of Server

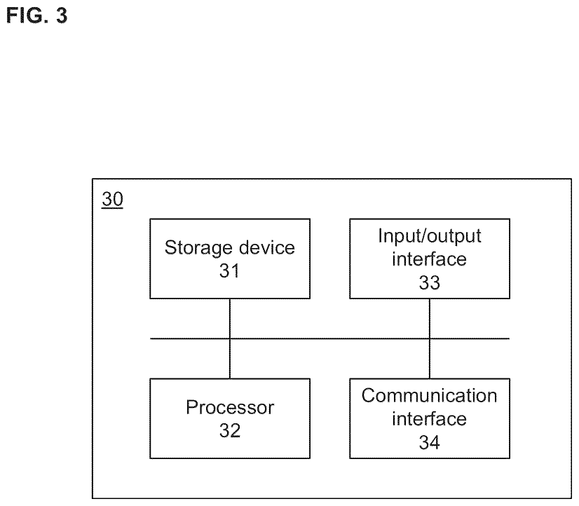

A configuration of the server will be described. FIG. 3 is a block diagram showing a configuration of the server of the present embodiment.

As shown in FIG. 3, the server 30 includes a storage device 31, a processor 32, an input/output interface 33, and a communication interface 34.

The storage device 31 is configured to store programs and data. The storage device 31 is, for example, a combination of a ROM, a RAM, and a storage.

The programs include, for example, the following programs.
OS program
Program of an application that executes information processing

The data includes, for example, the following data.
Database referred to in information processing
Result of executing information processing

The processor 32 is a computer that implements the functions of the server 30 by activating programs stored in the storage device 31. The processor 32 is, for example, at least one of the following.
CPU
GPU
ASIC
FPGA

The input/output interface 33 is configured to acquire information (such as a user instruction) from an input device connected to the server 30 and output information to an output device connected to the server 30.

The input device is, for example, a keyboard, a pointing device, a touch panel, or a combination thereof.

The output device is, for example, a display.

The communication interface 34 is configured to control communication between the server 30 and external apparatuses (such as the client apparatus 10).

### (1-3) Configuration of Wearable Device

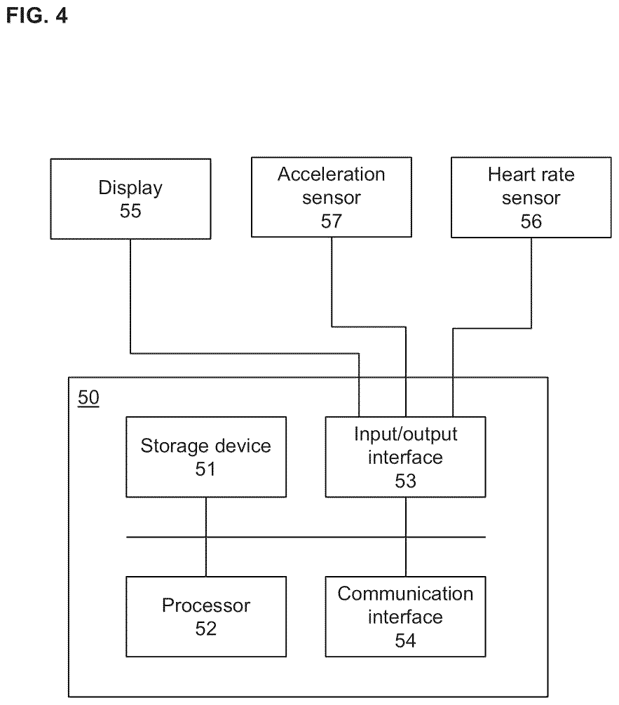

A configuration of the wearable device will be described. FIG. 4 is a block diagram showing a configuration of the wearable device according to the present embodiment.

As shown in FIG. 4, the wearable device 50 includes a storage device 51, a processor 52, an input/output interface 53, and a communication interface 54. The wearable device 50 is connected to a display 55, a heart rate sensor 56, and an acceleration sensor 57.

The storage device 51 is configured to store programs and data. The storage device 51 is, for example, a combination of a ROM, a RAM, and a storage.

The programs include, for example, the following programs.
OS program
Program for an application that executes information processing (e.g., a therapeutic application, a rehabilitation application, or a fitness application)

The data includes, for example, the following data.
Database referred to in information processing
Result of executing information processing

The processor 52 is a computer that implements the functions of the wearable device 50 by activating programs stored in the storage device 51. The processor 52 is, for example, at least one of the following.
CPU
GPU
ASIC
FPGA

The input/output interface 53 is configured to acquire information (such as a user instruction or a sensing result) from an input device connected to the wearable device 50 and output information (such as an image or a command) to an output device connected to the wearable device 50.

The input device is, for example, a heart rate sensor 56, an acceleration sensor 57, a microphone, a keyboard, a pointing device, a touch panel, or a combination thereof.

The output device is, for example, a display 55, a speaker, or a combination thereof.

The communication interface 54 is configured to control communication between the wearable device 50 and external apparatuses (such as the client apparatus 10).

Specifically, the communication interface 54 may include a module (e.g., a Bluetooth module) for communication with the client apparatus 10.

The display 55 is configured to display an image (still image or video). The display 55 is, for example, a liquid crystal display or an organic EL display.

The heart rate sensor 56 is configured to measure a heart rate and generate a sensing signal. As an example, the heart rate sensor 56 measures the heart rate using an optical measurement technique.

The acceleration sensor 57 is configured to detect acceleration.

### (2) Aspect of Embodiment

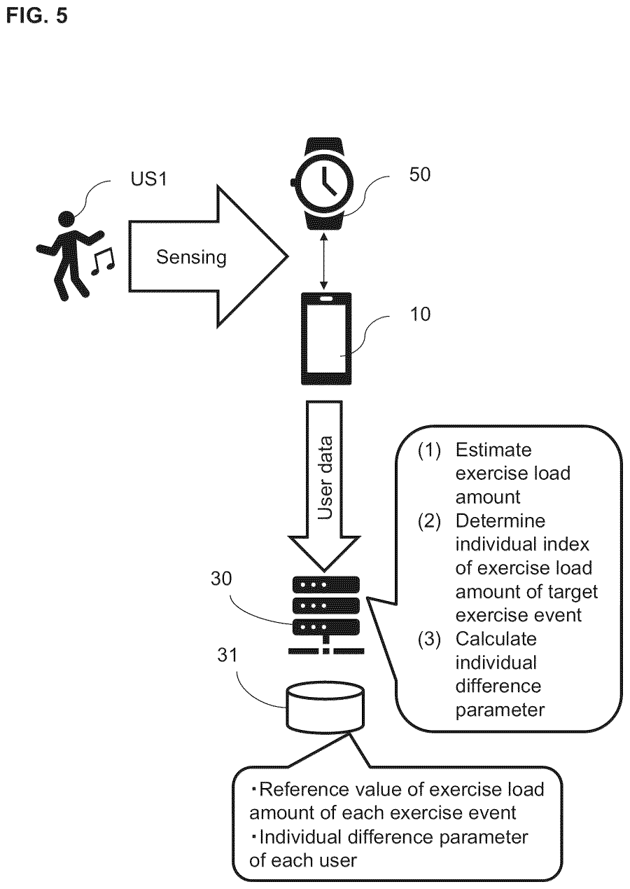

An aspect of the present embodiment will be described. FIG. 5 is an explanatory diagram of an aspect of the present embodiment.

As shown in FIG. 5, the client apparatus 10 and the wearable device 50 performs sensing of a user US1 during exercise. That is, the user selects and performs one of a plurality of available exercise events (hereinafter referred to as a “target exercise event”). The available exercise events are those for which a reference value (to be described later) of the exercise load amount corresponding to the exercise event is stored in the storage device 31 of the server 30. The user US1 is a person who receives exercise therapy, such as a participant of a (cardiac) rehabilitation program or an exercise instruction program. Although the example in FIG. 5 shows a case where the user US1 performs gymnastics exercise, the user US1 can perform any exercise (aerobic exercise or anaerobic exercise) among the plurality of available exercise events.

As an example, the camera 16 captures the appearance (for example, the whole body) of the user US1 during exercise from the front or diagonally forward at a distance of, for example, about 2 meters. The camera 16 may be installed at an appropriate height by a tripod or other height adjustment means. The depth sensor 17 measures a distance (depth) from the depth sensor 17 to each part of the user US1. Note that three-dimensional video data can be generated by combining, for example, video data (two dimensional) generated by the camera 16 and, for example, depth data generated by the depth sensor 17. The microphone 18 receives a sound emitted from the user US1 during exercise (e.g., a sound produced by breathing or speaking) and generates a sound signal.

The heart rate sensor 56 of the wearable device 50 measures the heart rate of the user US1 during exercise, and transmits the measurement result to the client apparatus 10. The acceleration sensor 57 measures the acceleration during the exercise of the user US1 and transmits the measurement result to the client apparatus 10.

The client apparatus 10 acquires various types of sensing data and performs analysis as necessary. As an example, the client apparatus 10 may analyze the physical condition of the user US1 during exercise with reference to the video data acquired from the camera 16. The client apparatus 10 may further refer to the depth data acquired from the depth sensor 17 in order to analyze the physical condition of the user US1 during exercise. The client apparatus 10 transmits, to the server 30, user data including at least one of the sensing data and the analysis result of the sensing data.

Based on the user data acquired from the client apparatus 10, the server 30 estimates the exercise load amount of the user while performing the target exercise event. The exercise load amount is estimated not once but at a plurality of time points for the target exercise event. Then, the server 30 performs a predetermined calculation on the exercise load amounts estimated at the plurality of time points to determine a personal index (to be described later) of the user US1 of the exercise load amount for the target exercise event. Furthermore, the server 30 calculates an individual difference parameter (to be described later) indicating the characteristics of the user US1 relating to the exercise tolerance (for example, whether the exercise load amount tends to be larger or smaller than that of a standard person) based on the determined individual index and the reference value (to be described later) of the exercise load amount for the target exercise event. For example, since the oxygen consumption during exercise depends on the amount of muscle, the exercise load amount varies from person to person even for the same exercise event, depending on how the muscles of the user US1 are built, the part to which the load is applied, and the like.

The server 30 stores the calculated individual difference parameter in the storage device 31. The server 30 can use the individual difference parameter to predict the exercise load amount when the user US1 performs a different exercise event than the target exercise event, or to estimate what exercise event the user (US1) should perform in order to achieve a desired exercise load amount. Furthermore, by continuously monitoring the individual difference parameter, it is possible to determine the trend in the body function of the user US1 (e.g., improvement, maintenance, or decline) or the rate of the change (e.g., rapid or not) and, for example, when the body function of the user US1 is rapidly declining, an alert indicating that an abnormality such as an aggravation of a heart disease such as a heart failure, a physical disorder, or a temporary deterioration of the physical condition is suspected can be output.

As described above, the information processing system 1 calculates the individual difference parameter representing the characteristics of the user US1 relating to the exercise tolerance, based on the sensing data of the user US1 during the target exercise event. Therefore, according to this information processing system 1, the individual difference parameter can be used as information for planning or guiding the exercise therapy provided to the user US1, or as a signal indicating the body function or physical condition of the user US1.

### (3) Databases

The databases of the present embodiment will be described. The following databases are stored in the storage device 31.

### (3-1) Exercise Event Database

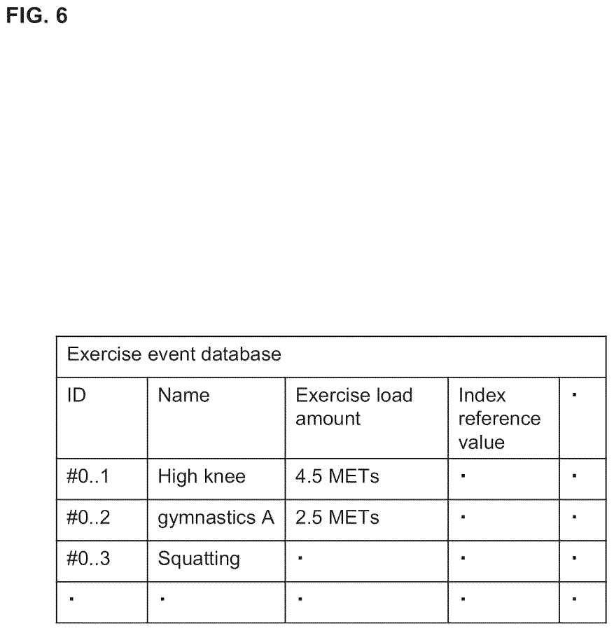

The exercise event database of the present embodiment will be described. FIG. 6 shows a data structure of the exercise event database of the present embodiment.

The exercise event database stores exercise event information. The exercise event information is information relating to exercise events that are recommended exercise events or target exercise event candidates (i.e., the aforementioned available exercise events). Here, in the present embodiment, the exercise events include, for example, gymnastics, body weight training, dancing, walking, running, a treadmill, and the like, which can be performed without using a device capable of adjusting the exercise load. These exercise events are varied because they include events that are performed in a standing position. In addition, in the present embodiment, the exercise loads of these exercise events can be adjusted through the form (e.g., range of motion of the part, degree of arm or leg opening, etc.), pace, number of reps, or time or number of breaks. However, the exercise events of the present embodiment may further include exercise events performed using a device capable of adjusting the exercise load, such as an ergometer, strength training with training equipment, and the like.

As shown in FIG. 6, the exercise event database includes an “ID” field, a “name” field, a “exercise load amount” field, and an “index reference value” field. The fields are associated with each other.

In the “ID” field, an exercise event ID is stored. The exercise event ID is information for identifying the exercise event corresponding to the record.

In the “name” field, exercise event name information is stored. The exercise event name information is information relating to the name of the exercise event corresponding to the record.

In the “exercise load amount” field, exercise load amount information is stored. The exercise load amount information is information relating to a standard exercise load amount of the exercise event corresponding to the record. The standard load amount is, for example, information relating to an exercise load amount when a person having a standard body function performs the corresponding exercise event. As an example, such an exercise load amount may be derived by actually measuring the exercise load amount (for example, average oxygen consumption) when a plurality of persons perform the corresponding exercise event through, for example, exhaled gas analysis, and statistically processing (for example, averaging) the measurement result, or may be acquired by referring to the exercise load amount set for the exercise event by a third party organization. The exercise load amount information may be managed in units finer than generally recognized exercise events. As an example, the exercise load amount information may be managed by each variation such as the form, pace, number of reps, or time or number of breaks for each exercise event. That is, even an exercise that is generally recognized as a single event (e.g., thigh raising) can be defined as a plurality of exercise events with slightly different exercise loads, by specifying the details of the form, pace, number of reps, and breaks. For example, an exercise that is generally recognized as a single event can be defined as a plurality of exercise events that differ by 0.2 METs from one another.

In the “index reference value” field, index reference value information is stored. The index reference value information is information relating to an indexed value of an exercise load amount when a person having a standard body function performs the exercise event corresponding to the record. As an example, such a value may be derived by, for example, actually measuring the exercise load amounts when a plurality of persons perform the corresponding exercise event in respective sections constituting the exercise event, calculating an individual index by applying the measurement results of the sections to a predetermined calculation formula for each person, and averaging the individual indexes among the persons. Here, the section is a constituent unit of the exercise event, and when the exercise event consists of a plurality of motion patterns, each motion pattern can correspond to a section. For example, when the exercise event is dancing, each choreography may correspond to a section. When the exercise event is squatting, the transition from standing to crouching and from crouching to standing can each correspond to a section. Note that the aforementioned exercise load amount information may be used as the index reference value information, and in this case, the “index reference value” field may be omitted.

### (3-2) User Profile Database

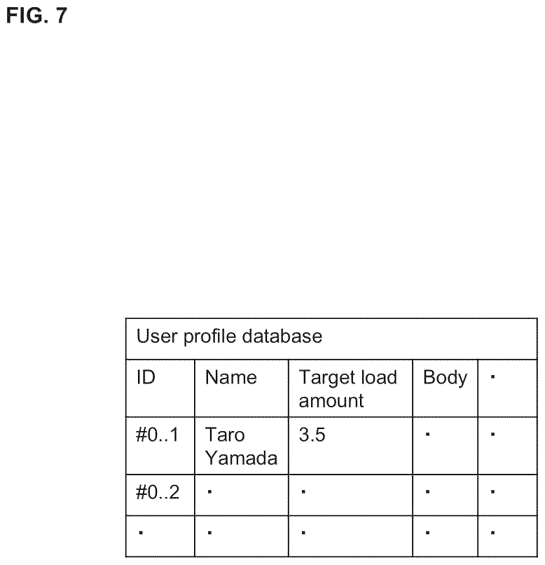

A user profile database of the present embodiment will be described. FIG. 7 shows a data structure of the user profile database of the present embodiment.

In the user profile database, user profile information is stored. The user profile information is information relating to a profile of a user of the information processing system 1 (that is, a person who performs exercise).

As shown in FIG. 7, the user profile database includes an “ID” field, a “name” field, a “target load amount” field, and a “body” field. The fields are associated with each other.

In the “ID” field, a user ID is stored. The user ID is information for identifying the user corresponding to the relevant record.

In the “name” field, user name information is stored. The user name information is information relating to the name (e.g., name, account name, etc.) of the user corresponding to the relevant record.

In the “target load amount” field, target load amount information (an example of the “predetermined exercise load amount”) is stored. The target load amount information is information relating to a target value of the exercise load amount (for example, an oxygen consumption, an energy consumption, a heart rate, or a combination thereof) set for the user corresponding to the record. As an example, the target load amount information is designated by a person (for example, a doctor) who plans or guides exercise therapy based on the result (for example, the oxygen consumption and heart rate at the anaerobic threshold (AT)) of measuring the exercise tolerance of the user by CPX when an ergometer is used, for example. However, CPX is not essential, and a target value may be designated at the discretion of the doctor. As another example, the target load amount information is determined by an algorithm based on the result of measuring the exercise tolerance of the user by, for example, CPX. For safer and more effective aerobic exercise, it is preferable to perform exercise at an intensity near the anaerobic threshold, and therefore, the target value of the exercise load amount is, for example, an exercise load amount corresponding to the anaerobic threshold, but is not limited thereto. Note that an ergometer is usually employed as the exercise event during CPX measurement and, for example, the oxygen consumption at the anaerobic threshold when a treadmill is employed is about 1.2 to 1.3 times the oxygen consumption at the anaerobic threshold when an ergometer is employed. This may be due to the fact that the total muscle mass used on the treadmill exceeds that used on the ergometer. Therefore, as a first example, the target load amount information may be a value obtained by correcting the target value based on the result measured by CPX when an ergometer is used to about 1.2 to 1.3 times or a value obtained by further subtracting a predetermined value (for example, 1 METs). As a second example, the target load amount information may be the target value based on the result measured by CPX when an ergometer is used, as it is. This can prevent the target value from becoming excessively high when an exercise event, such as an ergometer, which uses a relatively small total muscle mass, is selected. As a third example, the target value may be corrected using, for example, a coefficient corresponding to the total muscle mass to be used for each exercise event.

Note that the doctor may set an upper limit of the exercise load amount for the user (an example of exercise prescription). In this case, the user is not allowed to select an exercise event exceeding the upper limit of the prescribed exercise load amount. For exercise prescription, a user interface (UI) screen for exercise prescription may be displayed on the display of the terminal used by the doctor. The UI screen may include, for example, the following information.
CPX data of the user
Display areas of sample videos of a plurality of selectable exercise events
   Here, the display areas of the sample videos of the respective exercise events may be aligned and arranged by the exercise load information corresponding to each exercise event. For example, when 3.6 METs is recommended as the upper limit based on CPX data, a group of display areas of sample videos of exercise events corresponding to 3.4 METs, a group of display areas of sample videos of exercise events corresponding to 3.6 METs, and a group of display areas of sample videos of exercise events corresponding to 3.8 METs can be arranged on the UI screen.

When the doctor selects one of the display areas, the exercise load amount associated with the corresponding exercise event is set as the upper limit.

In addition, raising or lowering of the upper limit designated by the doctor through the exercise prescription may be performed by a health care provider under the supervision of the doctor during periodic (e.g., every two weeks) health care provider guidance or doctor's rounds.

In the “body” field, body information is stored. The body information is information relating to the body (function) of the user corresponding to the record. As an example, the body information may include information relating to the user's age, gender, weight, height, disease, etc.

In addition, the following information may be stored in the user profile database.
Information indicating a person who plans or guides exercise therapy of the user
Information indicating the user's doctor

### (3-3) Parameter Log Database

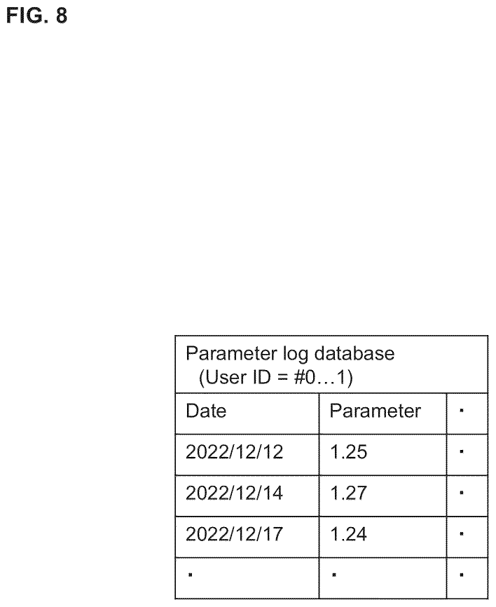

A parameter log database of the present embodiment will be described. FIG. 8 shows a data structure of the parameter log database of the present embodiment.

The parameter log database can be constructed for each user of the information processing system 1 (i.e., a person who performs exercise), for example. Alternatively, the parameter log database may be configured to store a record including information that can identify a user (e.g., a user ID).

In the parameter log database, parameter log information is stored. The parameter log information is information relating to a log of individual difference parameters calculated for the user.

As shown in FIG. 8, the parameter log database includes a “date” field and a “parameter” field. The fields are associated with each other.

In the “date” field, date information is stored. The date information is information relating to a date (or may be a date and time) on which the individual difference parameter of the relevant record was calculated.

In the “parameter” field, parameter information is stored. The parameter information is information relating to the value of the individual difference parameter of the relevant record.

### (4) Information Processing

Information processing of the present embodiment will be described.

### (4-1) Exercise Event Recommendation Processing

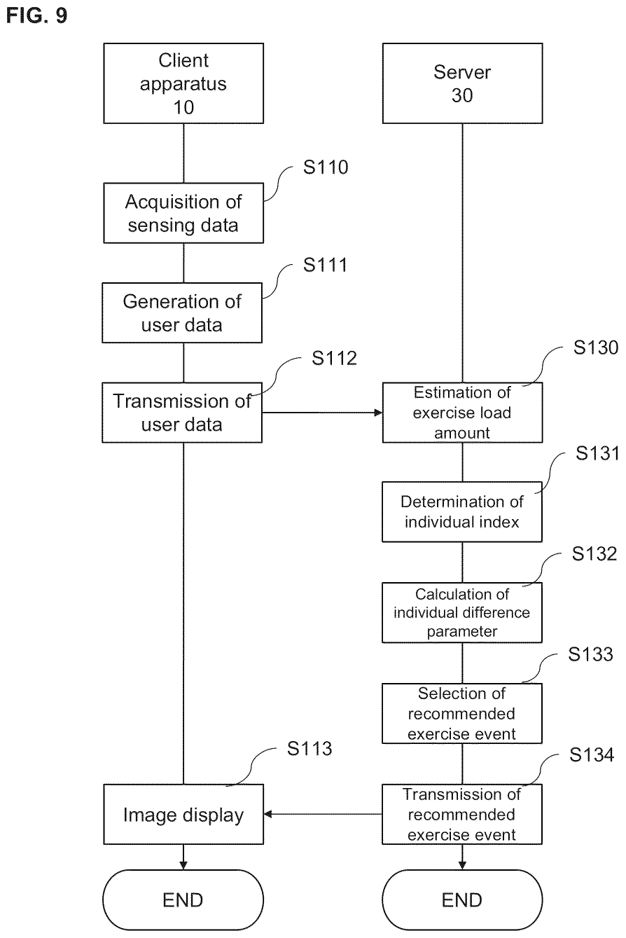

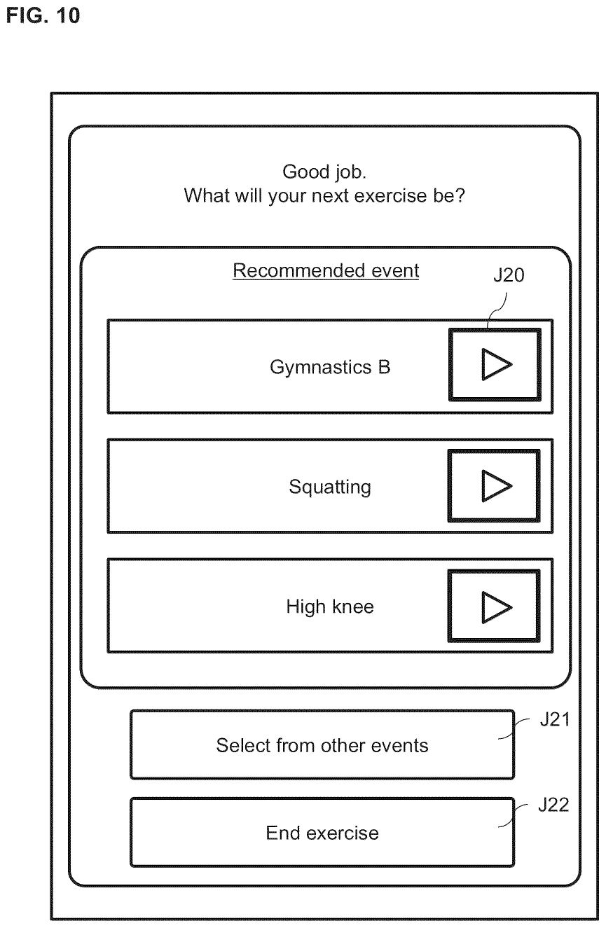

Exercise event recommendation processing according to the present embodiment will be described. FIG. 9 is a flowchart of the exercise event recommendation processing of the present embodiment. FIG. 10 shows a screen example displayed in the exercise event recommendation processing of the present embodiment.

The exercise event recommendation processing is started, for example, in response to the establishment of any of the following start conditions.
Exercise event recommendation processing has been invoked by other processing.
User or person who plans or guides exercise therapy of the user has performed an operation for invoking the exercise event recommendation processing
Client apparatus 10 has entered a predetermined state (e.g., a predetermined application has been started)
Appointed date and time has arrived
Predetermined time has elapsed since a predetermined event

As shown in FIG. 9, the client apparatus 10 executes acquisition of sensing data (S110).

Specifically, the client apparatus 10 may enable the operation of the camera 16 to start capturing a video of the user during exercise (hereinafter referred to as a “user video”). In addition, the client apparatus 10 may also enable the operation of the depth sensor 17 to start measuring the distance from the depth sensor 17 to each part of the user during exercise (hereinafter referred to as a “user depth”). The client apparatus 10 may enable the operation of the microphone 18 to start collecting sounds (e.g., sounds produced by the user's breathing or speaking (hereinafter referred to as “user sounds”)).

Furthermore, the client apparatus 10 may cause the wearable device 50 to start measuring the heart rate (hereinafter referred to as a “user heart rate”) with the heart rate sensor 56. In addition, the client apparatus 10 may enable any sensor (e.g., the acceleration sensor 19 or the acceleration sensor 57) of the client apparatus 10 or the wearable device 50.

Then, the client apparatus 10 acquires sensing data from each sensor.

Specifically, the client apparatus 10 acquires the sensing results generated by various sensors enabled in step S110. For example, the client apparatus 10 may acquire user video data from the camera 16, user depth data from the depth sensor 17, user sound data from the microphone 18, user heart rate data from the wearable device 50, and user acceleration data relating to the acceleration of the user (hereinafter referred to as “user acceleration”) from at least one of the acceleration sensor 19 and the wearable device 50.

After step S110, the client apparatus 10 executes generation of user data (step S111).

Specifically, the client apparatus 10 generates user data based on the sensing data acquired in step S110. The user data may include at least one of the following.
Data acquired in step S111 (e.g., user video data, user depth data, user sound data, user heart rate data, or user acceleration data)
Data obtained by processing the data acquired in step S111
Data (e.g., skeleton data, facial expression data, skin color data, respiration data, or a combination thereof) obtained by analyzing the user video data (or the user video data and the user depth data) acquired in step S111
Information capable of identifying the exercise event (target exercise event) that the user was performing in step S110
Information indicating the user's subjective evaluation of the exercise load amount of the target exercise event (hereinafter referred to as “subjective exercise intensity”)

After step S111, the client apparatus 10 executes transmission of the user data (S112).

Specifically, the client apparatus 10 transmits the user data acquired in step S111 to the server 30.

After step S112, the server 30 executes estimation of the exercise load amount (S130).

Specifically, the server 30 receives the user data transmitted by the client apparatus 10 in step S112. Based on the user data acquired from the client apparatus 10, the server 30 estimates the exercise load amount of the user while performing the target exercise event. The exercise load amount can be calculated as, for example, energy consumption (e.g., METs), oxygen consumption, exercise intensity based on heart rate (e.g., exercise intensity calculated by the Karvonen method), or a combination thereof. The server 30 may refer to the user profile information stored in the user profile database (FIG. 7) in order to estimate the exercise load amount.

As a first example of the estimation of the exercise load amount (S130), the server 30 estimates the exercise load amount of the user in each section constituting the target exercise by performing a Karvonen method calculation based on the heart rate of the user measured over a plurality of time points during the target exercise and the age of the user.

Note that the client apparatus 10 or the server 30 may analyze, for example, user video data (and user depth data if necessary) to identify to which section each sensing data is linked. Alternatively, when a fixed time is assigned to each section, for example, in the case of gymnastics or dancing, the client apparatus 10 or the server 30 may identify to which section each sensing data is linked based on the assignment of the time.

As a second example of the estimation of the exercise load amount (S130), the server 30 estimates the exercise load amount of the user in each section constituting the target exercise, using an estimation model to be described later. Specifically, the server 30 estimates the exercise load amount by applying an estimation model to input data based on user data (for example, skeleton data, facial expression data, skin color data, respiration data, heart rate data, or a combination thereof).

The server 30 may combine the first example and the second example.

Note that, since the estimated exercise load amount is not stable immediately after the start of the exercise (for 1 to 2 minutes, for example), the estimation of the exercise load amount may be omitted or the estimated exercise load amount may be discarded for a predetermined period from the start of the exercise. In other words, the server 30 may estimate the exercise load amount only when the state is determined as the plateau state.

After step S130, the server 30 executes determination of an individual index (S131).

Specifically, the server 30 performs a predetermined calculation using the exercise load amount estimated in step S130 to determine an individual index of the exercise load amount for the target exercise event for the user. As an example, the server 30 determines a representative value (for example, an average value, a median value, a mode value, a maximum value, a minimum value, a first quartile, or a third quartile) of the exercise load amount estimated for each section constituting the target exercise event as an individual index. As another example, the server 30 may determine the weighted sum of the amounts of exercise load estimated for the respective sections constituting the target exercise event as the individual index. The calculation formula of the individual index may be defined for each exercise event, or may be defined commonly for a plurality of exercise events.

After step S131, the server 30 executes calculation of an individual difference parameter (step S133).

Specifically, the server 30 refers to the exercise event database (FIG. 6) and acquires index reference value information of the target exercise event. Then, the server 30 calculates an individual difference parameter representing the characteristics of the user relating to the exercise tolerance based on the individual index determined in step S131 and the reference value of the exercise load amount for the target exercise event. The server 30 creates a record based on the calculated individual difference parameter and the calculation date (or calculation date and time), and adds the record to the user parameter log database (FIG. 8).

As a first example, the server 30 calculates the individual difference parameter by dividing the individual index by the reference value. In this case, the individual difference parameter corresponds to a prediction result of at how much ratio the exercise load amount of the user when the user performs the target exercise event or other exercise events will be greater or less than that of a standard person.

As a second example, the server 30 calculates the individual difference parameter by subtracting the reference value from the individual index. In this case, the individual difference parameter corresponds to a prediction result of by how much difference the exercise load amount of the user when the user performs the target exercise event or other exercise events will be greater or less than that of a standard person.

After step S132, the server 30 selects a recommended exercise event (S133).

Specifically, the server 30 refers to the user profile database (FIG. 7) and acquires the target load amount information of the user (which is determined based on the result of measuring the exercise tolerance of the user as described above). The server 30 selects an exercise event (hereinafter referred to as a “recommended exercise event”) suitable for the characteristics of the exercise tolerance of the user, based on the acquired target load amount information, the individual difference parameter calculated in step S132, and the exercise load amount information of each exercise event stored in the exercise event database (FIG. 6). The number of recommended exercise events may be one or more.

As a first example, the server 30 uses the individual difference parameter to correct the exercise load amount (hereinafter, referred to as a “standard load amount”) indicated by the exercise load amount information for each exercise event. For example, the server 30 obtains a corrected load amount by multiplying the standard load amount by the individual difference parameter or adding the individual difference parameter to the standard load amount. Then, the server 30 selects a recommended exercise event from the exercise events whose corrected load amounts do not exceed the target value of the exercise load amount of the user. For example, the server 30 may include, in the recommended exercise events, an exercise event whose corrected load amount is the maximum within a range equal to or less than the target value. Note that, in selecting the recommended exercise event, the server 30 may use a value obtained by adding or subtracting a margin to or from the target value, or a value obtained by multiplying the target value by a positive coefficient different from 1, instead of the target value. Alternatively, in selecting the recommended exercise event, the server 30 may use a value obtained by adding or subtracting a margin to or from the corrected load amount, or a value obtained by multiplying the corrected load amount by a positive coefficient different from 1, instead of the corrected load amount.

As a second example, the server 30 corrects the target value of the exercise load amount of the user by the individual difference parameter. For example, the server 30 obtains a corrected target value by dividing the target value by the individual difference parameter or by subtracting the individual difference parameter from the target value. Then, the server 30 selects a recommended exercise event from the exercise events whose standard load amount does not exceed the corrected target value. For example, the server 30 may include, in the recommended exercise events, an exercise event whose standard load is the maximum within a range equal to or less than the corrected target value. Note that, in selecting the recommended exercise event, the server 30 may use a value obtained by adding or subtracting a margin to or from the corrected target value, or a value obtained by multiplying the corrected target value by a positive coefficient different from 1, instead of the corrected target value. Alternatively, in selecting the recommended exercise event, the server 30 may use a value obtained by adding or subtracting a margin to or from the standard load amount, or a value obtained by multiplying the standard load amount by a positive coefficient different from 1, instead of the standard load amount.

After step S133, the server 30 executes transmission of the recommended exercise event information (S134).

Specifically, the server 30 transmits to the client apparatus 10 information relating to the recommended exercise event selected in step S133 (hereinafter referred to as “recommended exercise event information”). The recommended exercise event information may be, for example, information for identifying the recommended exercise event or screen information for displaying the recommended exercise event.

After step S134, the client apparatus 10 executes screen display (S113).

Specifically, the client apparatus 10 receives the recommended exercise event information transmitted by the server in step S134. The client apparatus 10 displays a screen based on the recommended exercise event information on the display 21.

For example, the client apparatus 10 displays the screen of FIG. 10 on the display 21. The screen of FIG. 10 includes objects J20 to J23.

Object J20 displays recommended exercise events. In addition, object J20 receives a user instruction to start a recommended exercise event or a user instruction to play a demonstration video of a recommended exercise event. When object J20 is selected, the client apparatus 10 may re-execute the exercise event recommendation processing of the present embodiment with the exercise event corresponding to the object J20 as a new target exercise event, or may play a demonstration video of the exercise event.

Object J21 receives a user instruction to select an exercise event other than the recommended exercise events. When object J21 is selected, the client apparatus 10 may display, for example, a list of exercise events other than the recommended exercise events, and receive a user instruction to select an exercise event. In response to receiving such a user instruction, the client apparatus 10 may re-execute the exercise event recommendation processing of the present embodiment with the selected exercise event as a new target exercise event, or may play a demonstration video of the exercise event.

Object J22 receives a user instruction to end the exercise. When object J22 is selected, the client apparatus 10 ends the exercise event recommendation processing of the present embodiment.

The client apparatus 10 may end the exercise event recommendation processing after step S113 (FIG. 9).

### (4-2) Parameter Monitoring Processing

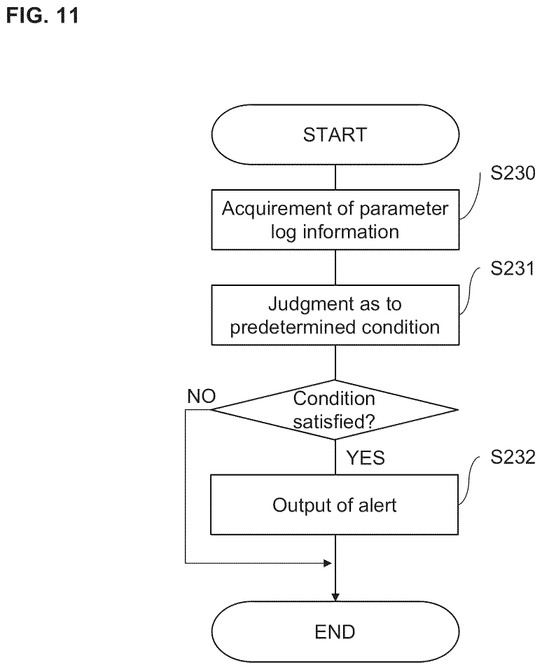

Parameter monitoring processing of the present embodiment will be described. FIG. 11 is a flowchart of the parameter monitoring processing of the present embodiment.

The parameter monitoring processing of the present embodiment may be started, for example, each time the exercise event recommendation processing (FIG. 9) of the present embodiment (particularly, the calculation of the individual difference parameter (S133)) is executed, or may be repeatedly executed in a predetermined cycle for each user.

As shown in FIG. 11, the server 30 performs acquisition of parameter log information (S230).

Specifically, the server 30 refers to the parameter log database of a user for which the processing is performed and acquires parameter log information.

After step S230, the server 30 executes judgment as to a predetermined condition (S231).

Specifically, the server 30 judges whether or not the predetermined condition is met for the parameter log information acquired in step S230. For example, the predetermined condition may include at least one of the following.
The representative value of a predetermined number of recent individual difference parameters (which may be one or more) exceeds a threshold (i.e., the user is predicted to have an excessively large exercise load amount compared to a standard person).
The representative value of the individual difference parameters over the most recent predetermined period exceeds a threshold (i.e., the user is predicted to have an excessively large exercise load amount compared to a standard person).
The rate of increase in the individual difference parameter about a predetermined number of recent individual difference parameters exceeds a threshold (i.e., the user is predicted to have a rapidly increasing exercise load amount for the equivalent exercise event).
The rate of increase in the individual difference parameter over the most recent predetermined period exceeds a threshold (i.e., the user is predicted to have a rapidly increasing exercise load amount for the equivalent exercise event).

When it is judged in step S231 that the predetermined condition is met, the server 30 executes output of an alert (S232).

Specifically, the server 30 transmits an alert to the client apparatus 10 of the user or a predetermined terminal. The predetermined terminal may be a terminal of a person designated by the user (e.g., a family member), a terminal of the user's doctor, or a terminal of a person who has planned or guided the user's exercise therapy.

The alert may include, for example, at least one of the following types of information.
A predetermined number of most recent individual difference parameters
Individual difference parameters over the most recent predetermined period.
The rate of increase in the individual difference parameter about a predetermined number of most recent individual difference parameters.
The rate of increase in the individual difference parameter over the most recent predetermined period
A message (e.g., text, image, sound, or a combination thereof) indicating that the user is suspected of having an aggravation of the cardiac disease
A message indicating that the user is suspected of having a physical disorder
A message indicating that the user is suspected of having temporary deterioration of the physical condition

The output destination of the alert (the client apparatus 10 of the user or a predetermined terminal) presents information based on the alert received from the server 30 through an image, sound, vibration, or a combination thereof.

After step S232, the server 30 ends the parameter monitoring processing of the present embodiment.

When it is judged that the predetermined condition is not met in step S231, the server 30 skips the output of an alert (S232) and ends the parameter monitoring processing of the present embodiment.

### (5) Summary

As explained above, the server 30 in the present embodiment estimates the exercise load amount when the user is performing the target exercise event based on the sensing data relating to the user, and performs a predetermined calculation on the exercise load amount to determine the individual index of the exercise load amount for the target exercise event for the user. The server 30 calculates an individual difference parameter representing the characteristics of the user relating to the exercise tolerance, based on the individual index and the reference value of the exercise load amount for the target exercise event. Therefore, the individual difference parameter can be used as information for planning or guiding exercise therapy to be provided to the user (which may include prescriptions), or as a signal indicating the body function or physical condition of the user.

The server 30 may select a recommended exercise event suitable for the exercise tolerance of the user from a plurality of exercise events based on the standard exercise load amounts of the plurality of exercise events, the individual difference parameter of the user, and the result of measuring the exercise tolerance of the user, and output information indicating the recommended exercise event. As a result, it is possible to recommend an exercise event not only by considering matching of the result of measuring the exercise tolerance of the user with the standard exercise load amounts of exercise events, but also the characteristics relating to the exercise tolerance of the user.

The server 30 may select a recommended exercise event so that the corrected exercise load amount obtained by correcting the standard exercise load amounts of the plurality of exercise events using the individual difference parameter of the user do not exceed a predetermined exercise load amount. Thus, it is possible to recommend an exercise event whose exercise load amount when the user actually performs the exercise event is estimated not to exceed the predetermined exercise load amount.

The server 30 may select recommended exercise events so as to include the exercise event whose corrected exercise load amount obtained by correcting the standard exercise load amount of each of the plurality of exercise events using the individual difference parameter of the user is the greatest without exceeding the predetermined exercise load amount. Thus, it is possible to recommend an exercise event whose exercise load amount when the user actually performs the exercise event is estimated not to exceed the predetermined exercise load amount and is estimated to be near the predetermined exercise load amount.

The predetermined exercise load amount may be determined in accordance with an exercise load amount designated by a person or algorithm that plans or guides exercise therapy of the user based on the result of measuring the exercise tolerance of the user. As a result, it is possible to recommend an exercise event suitable for the exercise load amount determined to be suitable for the user from a medical standpoint.

The server 30 may output an alert when the individual difference parameter of the user satisfies a predetermined condition. This allows the user himself/herself or a person concerned (e.g., family member or doctor in charge) to grasp changes in the body function or physical condition of the user at an early stage.

The individual difference parameter of the user may have a value corresponding to an excess of the individual index of the user over the reference value of the exercise amount for the target exercise event, and the predetermined condition may be that the excess exceeds a threshold. This allows the user himself/herself or a person concerned to grasp at an early stage that an abnormality such as an aggravation of a heart disease, a physical disorder, or a temporary deterioration of the physical condition of the user is suspected.

### (6) Estimation Model

As described above, the server 30 may estimate the exercise load amount using an estimation model. In this case, the estimation model corresponds to a trained model created by supervised learning using a training data set to be described below, a derivative model or a distilled model of the trained model. The estimation model may be built for each exercise event, or may be built in common for a plurality of exercise events.

### (6-1) Training Data Set

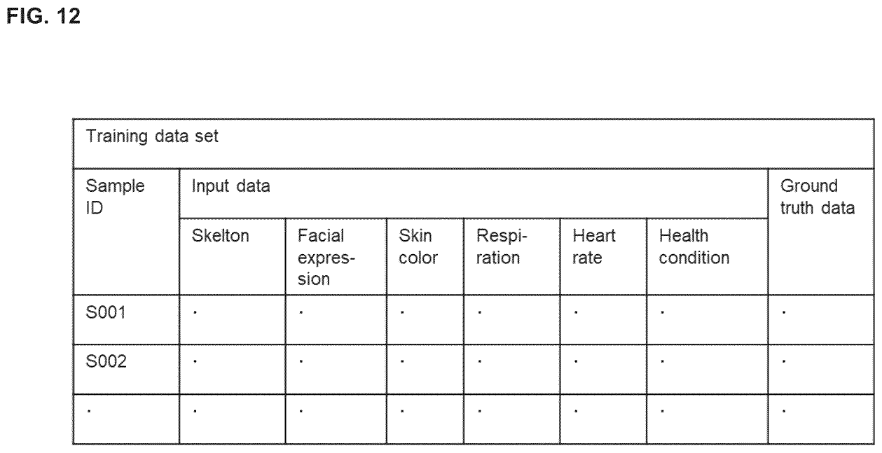

The training data set that can be used for supervised learning to build the estimation model. FIG. 12 shows a data structure of the training data set that can be used in the present embodiment.

As shown in FIG. 12, the training data set includes a plurality of training data. The training data is used for training or evaluating a model to be trained (hereinafter referred to as a “target model”). The training data includes a sample ID, input data, and ground truth data.

The sample ID is information for identifying training data.

The input data is data that is input to the target model during training or evaluation. The input data corresponds to an example used when training or evaluating the target model. As an example, the input data is data relating to the body condition of the subject during exercise (i.e., relatively dynamic data) and data relating to the health condition of the subject (i.e., relatively static data). At least a part of the data relating to the body condition of the subject is acquired by analyzing the physical condition of the subject with reference to the subject video data (or the subject video data and the subject depth data).

The subject video data is data relating to a subject video that shows the subject during exercise. The subject video data can be acquired, for example, by capturing the appearance (for example, the whole body) of the subject during a test relating to exhaled gas (for example, CPX test) from the front or diagonally forward (for example, 45 degrees forward) with a camera (for example, a camera mounted on a smartphone).

The subject depth data is data relating to the distance (depth) from the depth sensor to each part of the subject during exercise. The subject depth data can be acquired by operating the depth sensor when the subject video is captured.

The subject may be the same person as the user for whom the exercise load amount is estimated based on the exercise tolerance during the operation of the information processing system 1, or may be a different person from the user. By making the subject and the user the same person, the target model may learn the personality of the user and improve estimation accuracy. On the other hand, allowing the subject to be a person different from the user has the advantage of facilitating the enrichment of the training data set. The subject may be composed of a plurality of persons including the user or a plurality of persons not including the user.

In the example of FIG. 12, the input data includes skeleton data, facial expression data, skin color data, respiration data, heart rate data, and health condition data.

The skeleton data is data (for example, a feature) relating to the skeleton of the subject during exercise. The skeleton data includes, for example, data relating to the velocity or acceleration of each part of the subject (which may include data relating to a change in a part of a muscle used by the subject or a fluctuation in the body sensation of the subject). The skeleton data can be acquired by analyzing the skeleton of the subject during exercise with reference to the subject video data (or the subject video data and the subject depth data). As an example, Vision, which is an SDK for iOS (registered trademark) 14, or other skeleton detection algorithms (e.g., OpenPose, PoseNet, MediaPipe Pose) are available for the skeletal analysis. Alternatively, the skeleton data for the training data set can be acquired by, for example, causing the subject to exercise with a motion sensor attached to each part of the subject.

The result of skeleton detection can be used for quantitative or qualitative assessment of exercise, or a combination thereof. As a first example, the result of the skeleton detection can also be used to count the number of reps. As a second example, the result of skeletal sensing can be used to evaluate the adequacy of the form of the exercise or the load exerted by the exercise. For example, when the exercise event is squatting, the result of skeleton detection can be used to evaluate whether the knees are too far forward and in dangerous form, or whether the hips are firmly and deeply lowered so that sufficient load is exerted.

The facial expression data is data (e.g., a feature) relating to the facial expression of the subject during exercise. The facial expression data can be analyzed by applying an algorithm or a trained model to the subject video data. Alternatively, the facial expression data for the training data set can be acquired by, for example, labeling by a person who has viewed the subject video.

The skin color data is data (for example, a feature) relating to the skin color of the subject during exercise. The skin color data can be analyzed by applying an algorithm or a trained model to the subject video data. Alternatively, the skin color data for the training data set can be acquired by, for example, labeling by a person who has viewed the subject video.

The respiration data is data (e.g., a feature) relating to the respiration of the subject during exercise. The respiration information relates to, for example, the number of breaths per unit time or respiratory pattern. The respiratory pattern may include at least one of the following.
Number of ventilations
Ventilation volume
Ventilation rate (i.e., ventilation volume or number of ventilations per unit time)
Ventilation acceleration (i.e., time derivative of ventilation rate)
Carbon dioxide emission concentration
Carbon dioxide emissions (VCO2)
Oxygen uptake concentration
Oxygen uptake (VO2)

The data relating to the respiratory pattern may include data, such as a gas exchange ratio R (=VCO2/VO2), that can be calculated based on a combination of the above-described types of data.

The respiration data can be acquired, for example, by analyzing the skeleton data. As an example, the following items can be analyzed from the skeleton data.
Movement (spreading) of the shoulders, chest (which may include the lateral chest), abdomen, or a combination thereof
Inhalation time
Exhalation time
Degree of use of accessory respiratory muscle

The respiration data for the training data set can be obtained, for example, from the result of the test relating to the exhaled gas performed on a subject during exercise. Details of the exhaled gas test that can be performed on a subject during exercise will be discussed below. Alternatively, the number of ventilations, ventilation volume, ventilation rate, or ventilation acceleration in the respiration data for the training data set can be acquired from the result of a respiratory function test (for example, a pulmonary function test or a lung capacity test) performed on the subject during exercise. In this case, the instrument used in the respiratory function test is not limited to a medical instrument, and a commercially available test instrument may be used.

The heart rate data is data (e.g., a feature) relating to the heart rate of the subject during exercise. The heart rate data can be obtained, for example, by analyzing the subject video data or an analysis result thereof (e.g., skin color data). Alternatively, the heart rate data for the training data set may be obtained, for example, from the result of the test relating to the exhaled gas together with the respiration data to be described later. The subject's heart rate data for the training data set can also be obtained by causing the subject to perform the exercise with a heart rate sensor or electrodes for ECG monitoring attached thereto.

The health condition data is data relating to the health condition of the subject. The health condition data can be obtained in a variety of ways. The health condition data of the subject may be obtained before, during, or after the subject's exercise. The health condition data of the subject may be acquired based on a report from the subject or his or her doctor, may be obtained by extracting information linked to the subject in a medical information system, or may be obtained via an application (for example, a healthcare application) of the subject.

The health condition includes at least one of the following.
Age
Sex
Height
Body weight
Body fat percentage
Muscle mass
Bone mineral density
History of present illness
Medical history
Medication history
Surgical history
Life history (e.g., smoking history, alcohol history, activities of daily living (ADLs), frailty score, etc.)
Family history
Result of respiratory function test
Test result other than respiratory function test (e.g., blood test, urine test, electrocardiography (including Holter EKG), echocardiography, X-ray, CT scan (including cardiac morphological CT, and coronary CT), MRI scan, nuclear medicine test, PET scan, etc.)
Data obtained during cardiac rehabilitation (including Borg index)

The ground truth data is data corresponding to the ground truth of the corresponding input data (example). The target model is trained (supervised learning) to produce an output closer to the ground truth data in response to the input data. As an example, the ground truth data represents an exercise load amount.

The exercise load amount is an index to quantitatively assess the exercise load. The exercise load can be expressed numerically using at least one of the following.
Energy (calorie) consumption
Oxygen consumption
Heart rate

The ground truth can be obtained, for example, from the result of the test relating to exhaled gas performed on a subject during exercise. A first example of the test relating to exhaled gas is a test (typically a CPX test) performed while a subject wearing an exhaled gas analyzer is performing progressive-load exercise (e.g., ergometer). A second example of the test relating to the exhaled gas is a test performed while a subject wearing an exhaled gas analyzer is performing exercise with a load amount that is constant or changeable at any time (e.g., body weight exercise, gymnastics, strength training).

Alternatively, the ground truth data can be obtained from the result of a test other than the exhaled gas test performed on the subject during exercise. Specifically, the ground truth data can also be obtained from the result of a cardiopulmonary exercise load amount prediction test based on the measurement of lactate concentration in the sweat or blood of the subject during exercise. A wearable lactate sensor may be utilized to measure the lactate concentration of the subject.

It is also possible to build an estimation model for each of a plurality of health condition categories based on (at least a part of) the health condition of the subject. In this case, (at least a part of) the health condition of the user may be referred to in order to select an estimation model. In there further variations, the input data of the estimation model may be data that is not based on the health condition of the user, or data based on the health condition of the user and the user video.

### (7) Other Modifications

The storage device 11 may be connected to the client apparatus 10 via the network NW. Each input or output device may be built into the client apparatus 10. The storage device 31 may be connected to the server 30 via the network NW. Each input device or output device may be built into the wearable device 50.

An example has been shown in which the information processing system 1 of the embodiment is implemented by a client/server type system. However, the information processing system 1 of the embodiment can also be implemented by a peer-to-peer system or a stand-alone computer. As an example, the client apparatus 10 may estimate the exercise load amount.

Each step of the above-described information processing can be executed by either the client apparatus 10 or the server 30. As an example, instead of the client apparatus 10, the server 30 may acquire at least a part of the user data or the user skeleton data by analyzing the user video (or the user video and the user depth).

One or more steps of the above information processing may be performed using a trained model.

In the above example, the recommended exercise event is selected based on the standard exercise load amounts of the plurality of exercise events, the individual difference parameter of the user, and the result of measuring the exercise tolerance of the user. However, the server 30 may determine the content of the instruction for adjusting the exercise load amount for the exercise event selected by the user based on these types of information, and output information of the content of the instruction to the client apparatus 10. The content of the instruction can include, for example, the form (e.g., range of motion of a part, arm or leg opening, etc.), pace, number of reps, or time or number of breaks of the exercise event. Thus, for example, even when the exercise load amount when the user performs the exercise event selected by the user is estimated to exceed the above-described target value, it is possible to prevent the exercise load amount of the user from deviating from the target value by designating a form that makes the load lighter than the standard, setting the pace slower than the standard, reducing the number of reps from the standard, or increasing the time or the number of breaks. Alternatively, even when the exercise load amount when the user performs the exercise event selected by the user is estimated to fall below the above-described target value, it is possible to prevent the exercise load amount of the user from deviating from the target value by designating a form that makes the load heavier than the standard, setting the pace faster than the standard, increasing the number of reps from the standard, or reducing the time or the number of breaks.

In the above description, an example is shown in which the recommended exercise event is selected based on the standard exercise load amounts of the plurality of exercise events, the individual difference parameter of the user, and the result of measuring the exercise tolerance of the user. However, the server 30 may select the recommended exercise event further based on at least one of the pulse rate, the respiration rate, or the subjective exercise intensity of the user during exercise. Here, the subjective exercise intensity can be obtained by, for example, receiving an input of the Borg index from the user through the client apparatus 10 during break time. When at least one of the pulse rate, the respiration rate, or the subjective exercise intensity exceeds the upper limit value, the server 30 may select, as the recommended exercise event, an exercise event whose standard exercise load amount is lower than when at least one of the pulse rate, the respiration rate, or the subjective exercise intensity does not exceed the upper limit value. On the other hand, when at least one of the pulse rate, the respiration rate, or the subjective exercise intensity falls below the lower limit value, the server 30 may select, as the recommended exercise event, an exercise event whose standard exercise load amount is higher than when none of the pulse rate, the respiration rate, or the subjective exercise intensity falls below the lower limit value. However, when an upper limit of the exercise load amount is set for the user by the doctor, the exercise event corresponding to the exercise load amount exceeding the upper limit is not selected.

Further, the pulse rate, respiration rate, or subjective exercise intensity of the user during exercise may be used to adjust the exercise load amount of the next set when an exercise event consisting of a plurality of sets is performed. For example, when at least one of the pulse rate, the respiration rate, and the subjective exercise intensity exceeds the upper limit value, the server 30 may determine the content of the instruction (for example, the form, the pace, the number of reps, or the time or the number of breaks) so that the exercise load amount of the next set becomes lower than that of the current set, and output the information of the content of the instruction to the client apparatus 10. On the other hand, when at least one of the pulse rate, the respiration rate, and the subjective exercise intensity falls below the lower limit value, the server 30 may determine the content of the instruction so that the exercise load amount of the next set becomes higher than that of the current set, and output the content of the information to the client apparatus 10. However, when the upper limit of the exercise load amount is set for the user by the doctor, the content of the instruction corresponding to the exercise load amount exceeding the upper limit is not selected.

For example, when the average heart rate is higher than the target heart rate by 5 or more, or the subjective exercise intensity is 14 or more, the server 30 may determine the recommended exercise event or the content of the instruction so that the exercise load amount of the next set will be lower by 0.2 METs. When the average heart rate is higher than the target heart rate by 10 or more, or the subjective exercise intensity is 16 or more, the server 30 may determine the recommended exercise event or the content of the instruction so that the exercise load amount of the next set will be lower by 0.4 METs. In addition, when the average heart rate is lower than the target heart rate by 5 or more and the subjective exercise intensity is less than 10, the server 30 may determine the recommended exercise event or the content of the instruction so that the exercise load amount of the next set will be higher by 0.2 METs.

The client apparatus 10 may further execute the following processing, for example, during execution of the acquisition of sensing data (S110) (in other words, during exercise of the user). Specifically, the client apparatus 10 performs estimation relating to the skeleton of the user during exercise based on the sensing data. The client apparatus 10 then provides feedback to the user when the result of the estimation relating to the skeleton does not conform to at least one of the form and the pace defined for the exercise event being performed by the user (for example, the range of motion of a part is too narrow or too wide, the angle of the part deviates from the standard, or the pace is too fast or too slow). The feedback may include at least one of the following.
Light, sound, or voice (e.g., voice telling what the user is not doing correctly)
Display of an image (e.g., an image telling what the user is not doing correctly)
Vibration of the wearable device

Alternatively, the client apparatus 10 may recommend the user to redo the same exercise event or perform another exercise event when the number or frequency of times that the result of the estimation relating to the skeleton did not conform to at least one of the form or pace defined for the exercise event that the user is performing exceeds a threshold. In this case, the client apparatus 10 may omit the processes after the generation of the user data (S111).

The information processing system 1 may perform processing for measuring the oxygen consumption at the anaerobic threshold instead of the exercise event recommendation processing. This processing may be selected automatically, for example, during the first three days after the user starts exercise therapy, or may be selected in accordance with an instruction of the user or a person who plans or guides exercise therapy. In this mode, the information processing system 1 sequentially selects exercise events such that the exercise load increases by 0.2 METs until either the condition that the average heart rate is higher than the target heart rate by 5 or more or the condition that the subjective exercise intensity is 14 or more is satisfied, and the user performs the exercise. The information processing system 1 treats the oxygen consumption estimated for the exercise event performed immediately before the exercise event satisfying the above condition as the oxygen consumption at the anaerobic threshold of the user. In addition, the information processing system 1 may treat the exercise load amount corresponding to the exercise event by which the average heart rate closest to the target heart rate is obtained as the optimum exercise load amount.

In the above description, an example in which an alert is output when the individual difference parameter of the user satisfies a predetermined condition is described. However, not only the individual difference parameter but also various triggers may be used to output the alert. As an example, the server 30 stores data on exercise events performed by the user in the past and data on the heart rates, subjective exercise intensities, or respiration rates of the user during the exercise events. The server 30 may output an alert when the heart rate, the subjective exercise intensity, or the respiration rate of the user during exercise is increased (aggravated) by more than a threshold compared to the heart rate, the subjective exercise intensity, or the respiration rate of the user when the user performed the same exercise event in the past. Further, if the degree of user's anguish (hereinafter referred to as “degree of anguish”) is quantified based on, for example, facial expression data or skeleton data, it can be used as a trigger similar to the above-described heart rate, subjective exercise intensity, or respiration rate. In addition, the server 30 may use, as a trigger, an input relating to the appearance or aggravation of a subjective symptom of weight gain, shortness of breath, edema, fatigue, loss of appetite, or insomnia of the user in daily life, or a sensor input suggesting the appearance or aggravation of the symptom.

In the above description, an example is described in which the server 30 of the present embodiment estimates the exercise load amount of the user during the target exercise event, determines the individual index of the exercise load amount, and calculates the individual difference parameter. However, as another example, the server 30 obtains values of one or more variables (e.g., heart rate, respiration rate, subjective exercise intensity, or degree of anguish based on facial expression or skeleton (body movement)) relating to the physiological response of the user when the user is performing the target exercise event. For example, the server 30 can acquire these values from the client apparatus 10. Then, the server 30 performs a predetermined calculation on the acquired variable to determine the user's individual index of the variable for the target exercise event. Then, the server 30 estimates at least one of an exercise event that results in a predetermined exercise load amount when the user performs the exercise event or an exercise load amount when the user performs any of the exercise events, based on the determined individual index of the user and the reference value of the variable and the reference value of the exercise load amount for each of the plurality of exercise events including the target exercise event. The reference value of the variable can be calculated in the same manner as the reference value of the exercise load amount.

As a first example, the server 30 estimates the reference value of the exercise load amount of the exercise event corresponding to the reference value of the variable closest to the value of the individual index as the exercise load amount (Mt) when the user is performing the target exercise event, and estimates the exercise load amount (Mo) when the user performs another exercise event based on the reference value (Mrt) of the exercise load amount of the target exercise event and the reference value (Mro) of the exercise load amount of the other exercise event (for example, Mo=Mt*Mro/Mrt).

As a second example, the server 30 estimates the reference value of the exercise load amount of the exercise event corresponding to the reference value of the variable closest to the value of the individual index as the exercise load amount (Mt) when the user is performing the target exercise event, and estimates, as the exercise event that results in the predetermined exercise load amount (Mp) when the user performs the exercise event, an exercise event whose reference value (Mrp) of the exercise load amount is closest to a value (for example, Mrp=Mrt*Mp/Mt) based on the reference value (Mrt) of the exercise load amount of the target exercise event, the exercise load amount (Mt), and the predetermined exercise load amount (Mp) without exceeding the value.

In the above description, an example of capturing a user video with the camera 16 of the client apparatus 10 is described. However, the user video may be captured using a camera other than the camera 16. An example of measuring the user depth with the depth sensor 17 of the client apparatus 10 is shown. However, the user depth may be measured using a depth sensor other than the depth sensor 17.

In the above description, an example of measuring the heart rate of the user with the wearable device 50 is shown. However, the heart rate can be acquired by analyzing (e.g., remote photo-plethysmography (rPPG) analysis) the video data or the analysis result thereof (e.g., skin color data). The heart rate analysis may be performed by a trained model built utilizing a machine learning technique. Alternatively, the ECG monitor may measure the heart rate of the user by causing the user to perform an exercise with the ECG monitor electrode attached. In these variations, the user does not need to wear the wearable device 50 to measure the heart rate.

Instead of or in addition to the heart rate sensor 56 and the acceleration sensor 57, the wearable device 50 can include a sensor for measuring at least one of the following items.
Blood glucose level
Oxygen saturation

The measurement results of the sensors may be used to generate input data, estimate an exercise load amount or a ventilation index, present information based on the estimation result, or in other situations, as appropriate. As an example, the measurement result of the blood sugar level may be referred to evaluate the exercise load converted into the oxygen consumption or the energy consumption, for example. As another example, the measurement result of the acceleration can be used to determine the score of the exercise (for example, gymnastics) of the user, for example.

It is also possible to use acceleration data as a part of input data to the estimation model described in the present embodiment or modifications. Alternatively, the skeleton of the user may be analyzed with reference to the acceleration data. The acceleration data may be acquired by the acceleration sensor 19 or the acceleration sensor 57 at the time of capturing the user video, for example.

It is also possible to use oxygen saturation data as a part of input data to the estimation model described in the present embodiment or modifications. The oxygen saturation data can be acquired, for example, by having the user wear a wearable device provided with a sensor (for example, an optical sensor) capable of measuring the blood oxygen concentration or a pulse oximeter at the time of capturing the user video. The oxygen saturation data may be estimated by performing the rPPG analysis on the user video data, for example.

The information processing systems 1 of the present embodiment and each modification can be applied to a video game in which the progress of the game is controlled in accordance with the movement of the player's body. The video game may be a mini-game that can be played while the aforementioned therapeutic app, rehab app, or fitness app is running. As an example, the information processing system 1 may estimate the exercise load amount based on the exercise tolerance of the user during the game play, and determine one of the following in accordance with the result of the estimation (for example, a numerical value indicating the exercise load amount based on the exercise tolerance of the user). Thus, the effect of the video game on the health promotion of the user can be enhanced.
The quality (e.g., difficulty) or quantity of video game-related challenges (e.g., stages, missions, quests) provided to the user
The quality (e.g., type) or quantity of video game-related benefits (e.g., in-game currency, items, bonuses) provided to the user
Game parameters (e.g., score, damage) related to the progress of the video game

The information processing systems 1 of the present embodiment and each modification can be applied to a video game in which the progress of the game is controlled in accordance with the movement of the player's body. The video game may be a mini-game that can be played while the aforementioned therapeutic app, rehab app, or fitness app is running. As an example, the information processing system 1 estimates the skeleton of the user based on the user video during the game play. The estimation relating to the skeleton of the user may be further based on at least one of the user depth or the user acceleration in addition to the user video. Based on the result of the estimation relating to the skeleton of the user, the information processing system 1 evaluates the degree to which the posture of the user during exercise (for example, gymnastics) matches the ideal posture (model) The information processing system 1 may determine any one of the following in accordance with the result of this evaluation (for example, a numerical value indicating the conformity of the user's posture to the ideal posture). Thus, the effect of the video game on the health promotion of the user can be enhanced.
The quality (e.g., difficulty) or quantity of video game-related challenges (e.g., stages, missions, quests) provided to the user
The quality (e.g., type) or quantity of video game-related benefits (e.g., in-game currency, items, bonuses) provided to the user
Game parameters (e.g., score, damage) related to the progress of the video game

In addition to or instead of the microphone 18, a microphone of the wearable device 50 (a microphone included in or connected to the wearable device 50) may receive sound waves emitted by the user at the time of capturing the user video and generate sound data. The sound data may constitute input data to the estimation model described in the present embodiment or modifications. The sound generated by the user is, for example, at least one of the following.
Sound waves generated by the rotation of the user's legs (e.g., sounds generated by a pedal or a drive connected to a pedal)
Sounds produced by the user's breathing or speaking

In the above description, the CPX test is described as an example of the test relating to exhaled gas. In the CPX test, an incremental exercise load is applied to the test subject. However, it is not necessary to gradually increase the exercise load applied to the user at the time of capturing the user video. Specifically, the real-time exercise load amount can be estimated even in a state where a constant exercise load or an exercise load that can be changed at any time is applied to the user. For example, the exercise performed by the user may be body weight exercise, gymnastics, or strength training.

The functionality of the elements disclosed herein may be implemented using circuitry or processing circuitry which includes general purpose processors, special purpose processors, integrated circuits, ASICs (“Application Specific Integrated Circuits”), conventional circuitry and/or combinations thereof which are configured or programmed to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

The embodiment of the present invention has been described in detail above, but the scope of the present invention is not limited to the above-described embodiment. In addition, the above-described embodiment can be improved or modified in various ways without departing from the gist of the present invention. In addition, the above-described embodiment and modifications may be combined.

1. An information processing apparatus comprising processing circuitry configured to:
estimate a first exercise load amount when a first user is performing a target exercise event, based on sensing data relating to the first user;
determine a first individual index of the first user of an exercise load amount for the target exercise event by performing a predetermined calculation on the first exercise load amount; and
calculate a first parameter representing a characteristic of the first user relating to an exercise tolerance, based on the first individual index and a reference value of the exercise load amount for the target exercise event.

2. The information processing apparatus according to claim 1, wherein the processing circuitry is configured to:
perform estimation relating to a skeleton when the first user is performing the target exercise event, based on the sensing data relating to the first user; and
provide feedback to the first user when a result of the estimation relating to the skeleton does not conform to at least one of a form or a pace defined for the target exercise event.

3. The information processing apparatus according to claim 1, wherein the processing circuitry is configured to:
select a recommended exercise event suitable for the exercise tolerance of the first user from a plurality of exercise events, based on a standard exercise load amount of each of the plurality of exercise events, the first parameter, and a result of measuring the exercise tolerance of the first user; and
output information indicating the recommended exercise event.

4. The information processing apparatus according to claim 3, wherein the processing circuitry is configured to
select the recommended exercise event so that a corrected exercise load amount obtained by correcting the standard exercise load amount of each of the plurality of exercise events using the first parameter does not exceed a predetermined exercise load amount.

5. The information processing apparatus according to claim 4, wherein the processing circuitry is configured to
select the recommended exercise event so as to include an exercise event whose corrected exercise load amount obtained by correcting the standard exercise load amount of each of the plurality of exercise events using the first parameter is greatest without exceeding the predetermined exercise load amount.

6. The information processing apparatus according to claim 4, wherein
the predetermined exercise load amount is determined in accordance with an exercise load amount designated by a person or algorithm that plans or guides exercise therapy of the first user, based on the result of measuring the exercise tolerance of the first user.

7. The information processing apparatus according to claim 4, wherein the processing circuitry is configured to
output, when the first user performs a first exercise event after the first parameter is calculated, information relating to a form, pace, number of reps, or time or number of breaks of the first exercise event, based on a standard exercise load amount of an exercise load amount for the first exercise event, the first parameter, and the predetermined exercise load amount.

8. The information processing apparatus according to claim 1, wherein the processing circuitry is configured to
output an alert when the first parameter satisfies a predetermined condition.

9. The information processing apparatus according to claim 8, wherein
the first parameter has a value corresponding to an excess of the first individual index over a reference value of an exercise amount for the target exercise event, and
the predetermined condition is that the excess exceeds a threshold.

10. The information processing apparatus according to claim 3, wherein
the plurality of exercise events include an exercise event that can be performed without using a device capable of adjusting an exercise load.

11. The information processing apparatus according to claim 1, wherein
the reference value of the exercise load amount for the target exercise event is a representative value of exercise load amounts of the target exercise event measured for a plurality of persons.

12. An information processing apparatus comprising processing circuitry configured to:
acquire one or more variable values relating to a physiological response of a first user when the first user is performing a target exercise event;
determine a first individual index of the first user of the variable for the target exercise event by performing a predetermined calculation on the variable; and
estimate, based on the first individual index and a reference value of the variable and a reference value of an exercise load amount for each of the plurality of exercise events including the target exercise event, at least one of an exercise event that results in a predetermined exercise load amount when the first user performs the exercise event or an exercise load amount when the first user performs any of the exercise events.

13. A method for causing a computer comprising processing circuitry, wherein the processing circuitry executes:
estimating a first exercise load amount when a first user is performing a target exercise event, based on sensing data relating to the first user;
determining a first individual index of the first user of an exercise load amount for the target exercise event by performing a predetermined calculation on the first exercise load amount; and
calculating a first parameter representing a characteristic of the first user relating to an exercise tolerance, based on the first individual index and a reference value of the exercise load amount for the target exercise event.

14. A system comprising a first information processing apparatus and a second information processing apparatus, wherein
the first information processing apparatus comprises processing circuitry configured to:
acquire sensing data relating a first user from the second information processing apparatus;
estimate a first exercise load amount when the first user is performing a target exercise event, based on the sensing data;
determine a first individual index of the first user of an exercise load amount for the target exercise event by performing a predetermined calculation on the first exercise load amount; and
calculate a first parameter representing a characteristic of the first user relating to an exercise tolerance, based on the first individual index and a reference value of the exercise load amount for the target exercise event.
